SEQUENCE LISTING (1 sequence)

SEQ ID NO: 1 - DNA, 5959 bp, synthetic construct
  source 1..5959: mol_type = other DNA
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctaagctgttgtgaccgcttgctctagccagctatcgagttgtgaaccgatccatctagcaattggtctcgatctagcgataggcttcgatctagctatgtagaaacgccgtgtgctcgatcgcttgataaggtccacgtagctgctataattgcttcaacagaacatattgactatccggtattacccggcagatctttgtcgatcctaccatccactcgacacacccgccagcggccgaggaggtaatatgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcgtctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggacgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttcgtcgacgaaaacctgtacttccaaggtgactacaaggacgatgacgataagtggagccatccgcagtttgagaaatgaaagcttccgagctctcgaattcaaaggaggtacccaccatggggtaccgcgatatctacctcgaggtttctagaagttgtctcctcctgcactgactgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggca

(19) United States
(12) Patent Application Publication
(10) Pub. No.: US 2026/0256964 A1
(43) Pub. Date: Sep. 3, 2026

(54) Epitope-Independent Pretargeted Therapy of Cancers Using Bacteria

(71) Applicant: UNIVERSITY OF CINCINNATI, Cincinnati, OH (US)

(72) Inventors: Nalinikanth KOTAGIRI, West Chester, OH (US); Nabil SIDDIQUI, Cincinnati, OH (US)

(73) Assignee: University of Cincinnati, Cincinnati, OH (US)

(21) Appl. No.: 18/872,857

(22) PCT Filed: Jun. 7, 2023
(86) PCT No.: PCT/US2023/024750

§ 371 Date: Dec. 9, 2024

Related U.S. Application Data
(60) Provisional application No. 63/350,015, filed on Jun. 7, 2022.

(51) Int. Cl. A61K 51/12; A61K 51/04; C07K 14/245

(52) U.S. Cl. CPC A61K 51/1203; A61K 51/0453; C07K 14/245

(57) ABSTRACT

A genetically engineered Escherichia coli Nissle 1917 bacterium is provided. The genetically engineered bacterium comprises an overexpressed number of FyuA receptors. In one embodiment, the genetically engineered bacterium comprises the nucleotide sequence shown in SEQ ID NO:1. In another embodiment, the genetically engineered bacterium further comprises cell-associated 67Cu complexed to yersiniabactin (67Cu-YbT). In one embodiment, the genetically engineered bacterium further comprises cell-associated 64Cu complexed to yersiniabactin (64Cu-YbT).

### CROSS-REFERENCE TO RELATED APPLICATIONS

This application is a continuation of PCT Application No. PCT/US23/24750 filed Jun. 7, 2023, which claims the benefit of the filing date of, U.S. Patent Application Ser. No. 63/350,015, filed on Jun. 7, 2022, the disclosures of which are incorporated by reference herein in their entirety.

### STATEMENT REGARDING FEDERALLY FUNDED RESEARCH OR DEVELOPMENT

This invention was made with government support under R21GM137321 awarded by the National Institutes of Health. The government has certain rights in the invention.

### TECHNICAL FIELD

The present invention relates to pre-targeted therapies for cancer.

### BACKGROUND OF THE INVENTION

A plethora of novel chemotherapeutics, biologics, immune checkpoint inhibitors and eventually cell-therapies have been developed in the last two decades. However, many patients with cancer have progression refractory to conventional therapies. In addition, off-target toxicities present a formidable challenge in the curative and non-curative settings, particularly in patients with solid tumors. While radiotherapy has been used to treat solid cancers for more than a century, it has primarily involved administration of radiation from outside the body to kill malignant cells inside. Targeted radionuclide therapy (TRT) is an emerging therapeutic modality owing to its numerous favorable attributes over existing approaches. Unlike external beam radiation therapy, which is unable to address systemic metastases, TRT involves administration of specific radiopharmaceuticals that selectively bind to cancer cells. Once bound, the radioactive nuclides emit cytotoxic doses of a and B particles to the surrounding cancer cells. In addition, TRT facilitates visualization of the radiopharmaceutical probes by imaging techniques such as positron emission tomography (PET) and single-photon emission computed tomography, for confirming probe localization, and enables an image-guided theranostic approach to monitoring treatment outcomes.

Although TRT has proven to be effective when other standard approaches have failed, it should be noted that this radiotherapeutic modality has been employed only for cancers with available target epitopes. For instance, even though 227Th-conjugated anti-HER2/neu demonstrates excellent anti-tumor efficacy, it is unlikely to be effective in patients where the HER2 receptor undergoes endocytosis, and hence is not available on the cancer cell membrane for binding. Even when the target antigens are available, the specificity of the therapeutic probe has to be high to avoid delivery of cytotoxic radiation to non-cancerous cells that have a basal level of expression of the same epitopes, albeit not as high as the cancer cells. For example, a radiolabeled GD2—(a major ganglioside present in human neuroblastoma) targeting antibody, that demonstrates favorable tumor: non-tumor ratio in a pre-targeted approach, might still induce significant central nervous system toxicities to pediatric patients suffering from neuroblastoma due to shared expression of GD2 in vital normal tissues. Furthermore, in traditional TRT, since the emitted radiation originates from the cancer cell-bound radionuclide, the probability of killing cells depends on the number of target cells. In solid cancers, such as pancreatic cancer, with high density of extracellular matrix, fewer cancer cells potentially lead to a smaller fraction of the emitted energy deposited onto target cells. Therefore, a need still exists for alternative targeted and pre-targeted strategies to address shortcomings of the current TRT paradigm.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, a genetically engineered Escherichia coli Nissle 1917 bacterium is provided. The genetically engineered bacterium comprises an overexpressed number of FyuA receptors. In one embodiment, the genetically engineered bacterium comprises the nucleotide sequence shown in SEQ ID NO:1.

In another embodiment, the genetically engineered bacterium further comprises cell-associated ⁶⁷Cu complexed to yersiniabactin (⁶⁷Cu-YbT). In one embodiment, the genetically engineered bacterium further comprises cell-associated ⁶⁴Cu complexed to yersiniabactin (⁶⁴Cu-YbT). In another embodiment, a pharmaceutical composition is provided that comprises the genetically engineered bacterium described above and a pharmaceutically acceptable excipient.

In another aspect of the present invention, a method of providing targeted radionuclide therapy to subject with a tumor having cancer cells is provided. The method involves administering to the subject a bacterium that has been genetically engineered to have an overexpressed number of FyuA receptors. The bacterium also comprises at least one cell-associated copper radioisotope. In addition, the bacterium populates the tumor and delivers a cytotoxic dose of the radioisotope to the cancer cells.

In one embodiment of the method, the copper radioisotope is ⁶⁷Cu. In another embodiment of the method, the copper radioisotope is complexed to yersiniabactin (YbT). In one embodiment of the method, the bacterium is genetically engineered Escherichia coli Nissle 1917. In another embodiment of the method, the bacterium comprises the nucleotide sequence shown in SEQ ID NO:1.

In another aspect of the present invention, a method of imaging a tumor in subject with a tumor is provided. The method involves administering to the subject a bacterium that has been genetically engineered to have an overexpressed number of FyuA receptors. The bacterium further comprises at least one cell-associated copper radioisotope. Also, the bacterium populates the tumor. In addition, positron emission tomography (PET) imaging is conducted on the subject after the bacterium has populated the tumor.

In one embodiment of the method, the copper radioisotope is ⁶⁴Cu. In another embodiment of the method, the copper radioisotope is complexed to yersiniabactin (YbT). In one embodiment of the method, the bacterium is genetically engineered Escherichia coli Nissle 1917. In another embodiment of the method, the bacterium comprises the nucleotide sequence shown in SEQ ID NO:1.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention.

FIG. 1A is a schematic of a reaction mechanism depicting the complexation of ^{64/67}Cu by yersiniabactin (YbT).

FIG. 1B is a is a schematic highlighting FyuA-specific delivery of Cu-YbT radiopharmaceuticals by EcN-based pretargeted cancer theranostic platform.

FIG. 1C is an immunoblot confirming FyuA expression by engineered EcN.

FIG. 1D is a schematic of EcN constructs used for FIG. 1E.

FIG. 1E is a graph showing a quantitative (regression curve) assessment of FyuA overexpression.

FIG. 1F is a graph showing a dissociation curve of ⁶⁴Cu-YbT from EcN-fyuA↑.

FIG. 2A is a schematic showing BLI, PET/CT, and ex vivo BioD analyses depicting EcN-fyuA↑ localization in MC38 tumors and FyuA-specific retention of ⁶⁴Cu-YbT 1-day post intratumoral administration of bacteria.

FIG. 2B is a graph showing the general biodistribution of ⁶⁴Cu-YbT.

FIG. 2C is a graph showing the results of a qRT-PCR assay confirming presence of EcN-fyuA↑ in MC38 tumors 1- and 7-days post intratumoral administration of bacteria.

FIG. 2D is a schematic showing BLI, a graph showing ex vivo BioD, and a schematic showing PET/CT demonstrating the presence of bacteria in MC38 tumors 18-days after intratumoral injection.

FIG. 3A is a schematic of the in vivo therapeutic plan.

FIG. 3B is a pair of graphs showing tumor progression during the initial stages of therapy and survival curves of MC38 tumor-bearing C57BL6/J mice (n=4-8).

FIG. 3C is a pair of graphs showing 4T1 tumor-bearing BALB/cJ mice (n=4) in the four treatment regimens.

FIG. 4A is a graph showing flow cytometry analyses of tumor immune cell infiltrates for total immune cells (CD45+) 7 days after ⁶⁷Cu-YbT administration in MC38 tumor-bearing mice with or without probiotic administration.

FIG. 4B is a graph showing flow cytometry analyses of tumor immune cell infiltrates for total T cells 7 days after ⁶⁷Cu-YbT administration in MC38 tumor-bearing mice with or without probiotic administration.

FIG. 4C is a graph showing flow cytometry analyses of tumor immune cell infiltrates for CD4+ T cells 7 days after ⁶⁷Cu-YbT administration in MC38 tumor-bearing mice with or without probiotic administration.

FIG. 4D is a graph showing flow cytometry analyses of tumor immune cell infiltrates for Tregs (CD4+ CD25+FOXP3+) cells 7 days after ⁶⁷Cu-YbT administration in MC38 tumor-bearing mice with or without probiotic administration.

FIG. 4E is a graph showing flow cytometry analyses of tumor immune cell infiltrates for CD8+ T cells as a percent of total live cells 7 days after ⁶⁷Cu-YbT administration in MC38 tumor-bearing mice with or without probiotic administration.

FIG. 4F is a graph showing flow cytometry analyses of tumor immune cell infiltrates for CD8+T:T_{reg} ratio 7 days after ⁶⁷Cu-YbT administration in MC38 tumor-bearing mice with or without probiotic administration.

FIG. 5A is a graph showing liver function analyses of serum obtained from ⁶⁷Cu-YbT treated C57BL6/J mice.

FIG. 5B is a graph showing liver function analyses of serum obtained from ⁶⁷Cu-YbT untreated C57BL6/J mice.

FIG. 5C is a graph showing kidney function analyses of serum obtained from ⁶⁷Cu-YbT treated and untreated C57BL6/J mice.

FIG. 5D is a graph showing the weight of mice monitored every 2 days following administration of ⁶⁷Cu-YbT.

FIG. 6 is a schematic and SnapGene map of a fyuA expression plasmid.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, “cell-associated” means bound to, connected to, and included in a cell. Thus, in various exemplary embodiments, “associated with a cell” is associated with a cell (e.g., associated with a cell receptor) and/or associated with a cell structure and/or inside the outermost membrane of a cell.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid or solvent encapsulating material, involved in carrying or transporting the subject compound from one organ, or portion of the body, to another organ, or portion of the body. Each excipient must be “acceptable” in the sense of being compatible with the other ingredients of the formulation and not injurious to the patient.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

In one embodiment, the present invention takes the novel approach of using a bacterium for targeted radionuclide therapy (TRT). As shown by the data presented herein, this platform has immunomodulatory (increased anti-tumor CD8+ T cell population, decreased anti-inflammatory Treg population) properties. TRT is an emerging therapeutic modality for the treatment of various solid cancers. Current approaches rely on the presence of cancer-specific epitopes and receptors against which a radiolabeled ligand is systemically administered to specifically deliver cytotoxic doses of a and B particles to tumors. The present invention takes the novel approach of utilizing tumor-colonizing Escherichia coli Nissle 1917 (EcN) to deliver a bacteria-specific radiopharmaceutical to solid tumors in a cancer-epitope independent manner. In one embodiment, this microbe-based pretargeted approach leverages the siderophore-mediated metal uptake pathway to selectively concentrate copper radioisotopes, ⁶⁴Cu and ⁶⁷Cu, complexed to yersiniabactin (YbT) in the genetically modified bacteria. ⁶⁴Cu-YbT facilitates positron emission tomography (PET) imaging of the intratumoral bacteria, whereas ⁶⁷Cu-YbT delivers a cytotoxic dose to the surrounding cancer cells. PET imaging with ⁶⁴Cu-YbT reveals persistence and sustained growth of the bioengineered microbes in the tumor microenvironment. Survival studies with ⁶⁷Cu-YbT reveals significant attenuation of tumor growth and extends survival of both MC38 and 4T1 tumor-bearing mice harboring the microbes. Tumor response to this pre-targeted approach correlates with promising anti-tumor immunity, with noticeable CD8+ T:Treg cell ratio. The present invention offers a pathway to target and ablate multiple solid tumors independent of their epitope and receptor phenotype.

Prior studies using engineered EcN have relied on synthesizing and secreting therapeutic proteins in the tumor microenvironment, in the form of nanobodies, peptides, and enzymes. The present invention allows the use of engineered bacteria essentially as an “adapter” that can be incorporated in tumors, which carry the necessary receptors to attract a therapeutic radioisotope (⁶⁷Cu) with a high degree of selectivity and precision. In the examples below, it is demonstrated that instead of secreting proteins, the engineered bacteria can be utilized as a pre-targeted platform to attract systemically administered therapeutic radionuclides into the tumor microenvironment that is devoid of targetable biomarkers.

Besides its ability to deliver therapeutic molecules, EcN is also an excellent template for molecular imaging in vivo. In another embodiment of the present invention, EcN is noninvasively tracked via PET imaging, using a ⁶⁴Cu-labeled bacterial siderophore, yersiniabactin (YbT). The ⁶⁴Cu-YbT probe specifically accumulates in bacteria like EcN that express the ferric YbT uptake receptor (FyuA).

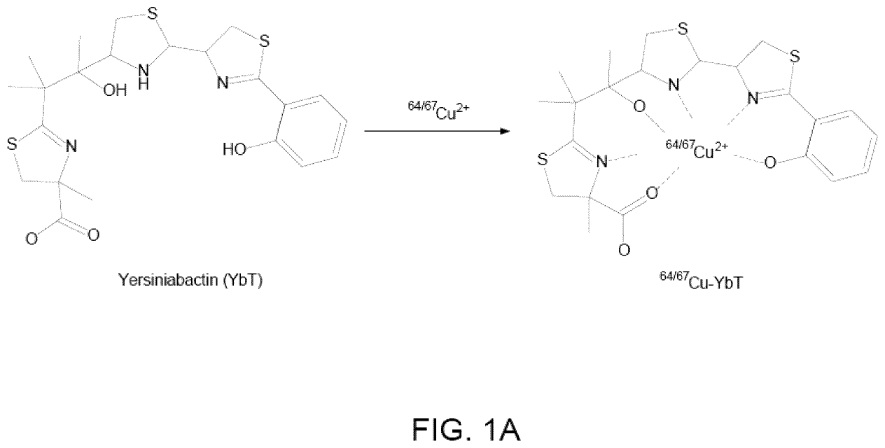

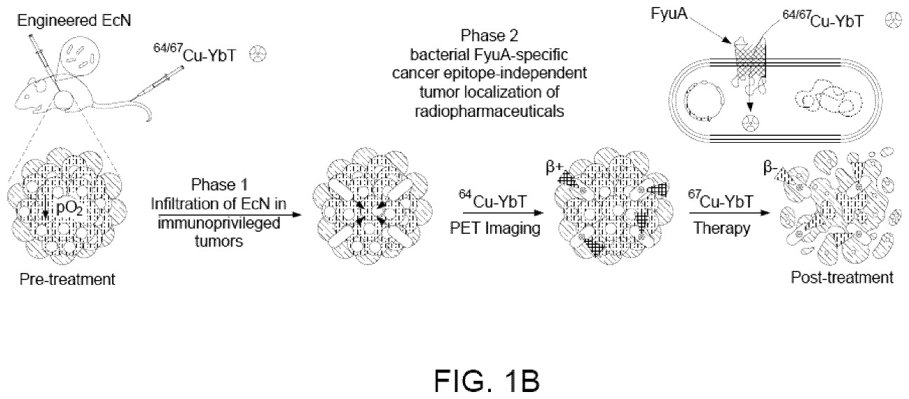

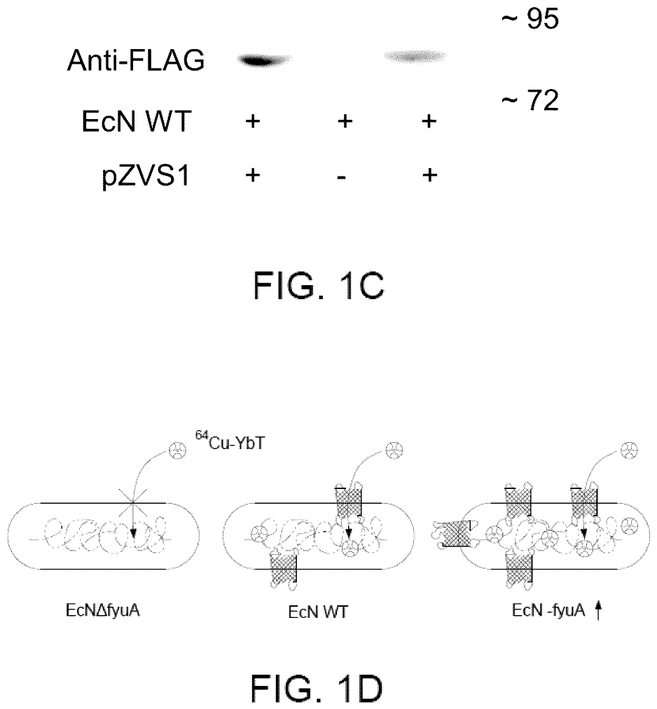

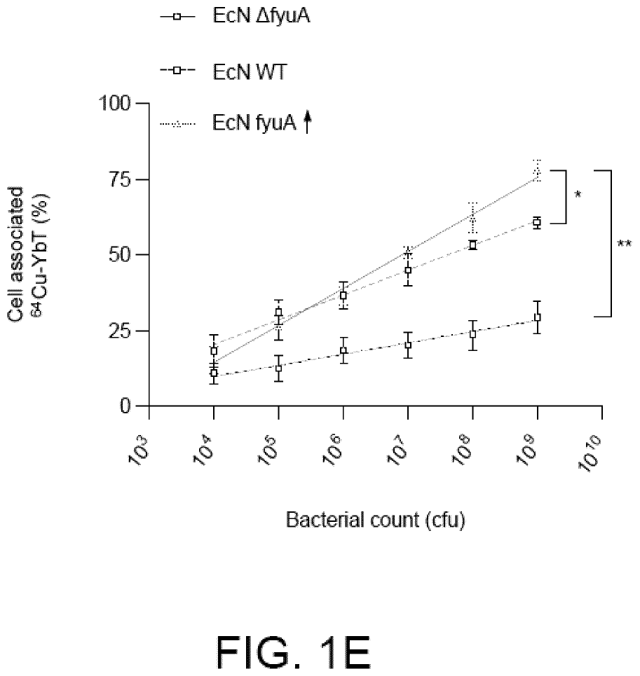

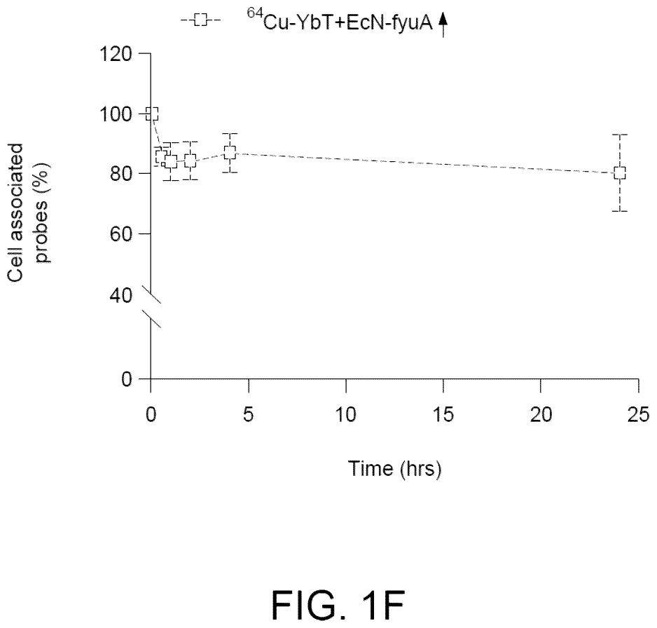

As discussed above, one embodiment of the present invention involves a therapeutic radionuclide, ⁶⁷Cu, ⁶⁷Cu-YbT that can be delivered to probiotic EcN localized in solid tumors and elicit a “bystander” cytotoxic effect on the cancer cells, irrespective of their biomarker profile. ⁶⁴Cu-YbT and ⁶⁷Cu-YbT can thus be used interchangeably to facilitate image-guided TRT of solid tumors (see FIGS. 1A and 1B).

Due to tumor heterogeneity, the biomarker profile of tumors is often indeterminate making it difficult to devise targeted strategies for imaging and therapy. Therefore, there is a need for novel targeted systems, like the present invention, that can be used to ablate multiple tumor types irrespective of their antigen and receptor phenotype. As part of the invention, E. coli Nissle was engineered to overexpress the metal uptake receptor, FyuA, on the bacterial cell-surface. The engineered bacterium binds to and internalizes its cognate probes ^{64/67}Cu-YbT with high specificity. The probes are highly switchable as the ligand (YbT) can chelate ⁶⁴Cu (imaging) and ⁶⁷Cu (therapy) radioisotopes. The engineered bacteria are not only able to populate the tumors, but also thrive and maintain sustainable growth kinetics in tumors, which can be tracked longitudinally using the ⁶⁴Cu-labeled probe. In addition, the present invention has found that FyuA can serve as a foreign epitope that can then be superimposed on the cancer cells in the tumor matrix, to attract and bind ⁶⁷Cu-labeled YbT to initiate therapy. Since the surface proteins are genetically encoded by the bacteria, the density and number of these artificial epitopes is amplified as a function of time and size of tumors. The examples presented herein also indicate that EcN-fyuA↑ sequestered ⁶⁷Cu-YbT from the systemic circulation and induced anticancer effects by modulating the local immune cell populations. The CD8+ T:Treg cell ratio was significantly higher in the tumor microenvironment following ⁶⁷Cu-YbT+EcN-fyuA↑ therapy.

### EXAMPLES

### Materials and Methods

Chemicals: All reagents were purchased from commercial sources as analytical grade and used without further purification. YbT was purchased from EMC Microcollections GmbH (Tuebingen, Germany). ⁶⁴Cu was obtained from Mallinckrodt Institute of Radiology, Washington University School of Medicine, while ⁶⁷Cu from Idaho Accelerator Center, Idaho State University.

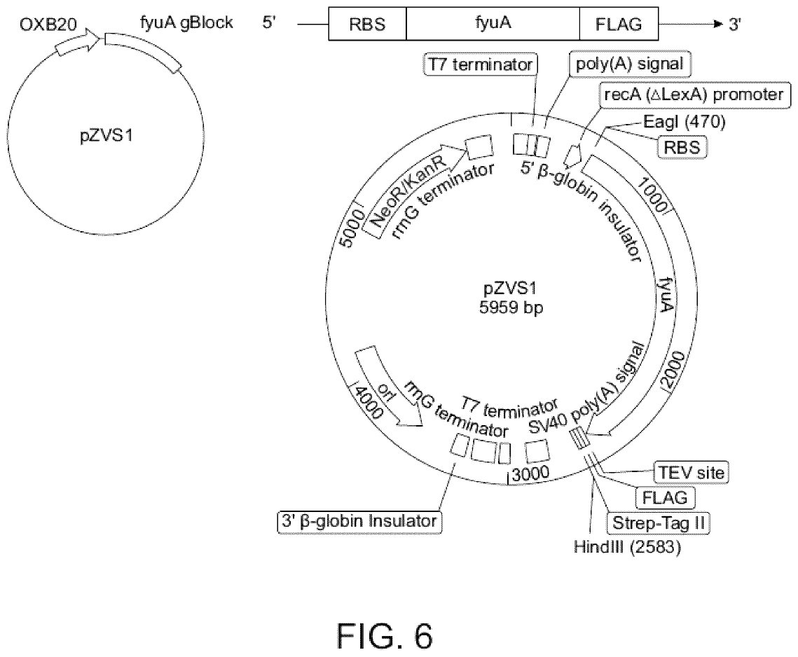

Bacterial Strain and Plasmids: E. coli Nissle (Mutaflor) was used for the examples. Plasmids were constructed using standard restriction enzyme-mediated cloning methods. To construct pZVS1, the fyuA gBlock (FIG. 6) flanked by appropriate restriction sites was purchased from Integrated DNA Technologies. This sequence was cloned into pSF-OXB20 (Millipore Sigma) using EagI and HindIII sites. Following plasmid construction at each stage, the sequences were verified via Sanger sequencing before EcN was transformed. FyuA KO was performed in EcN using the standard red recombinase method as previously described. For BLI, EcN-fyuA↑ was transformed with pGEN-luxCDABE (Addgene plasmid #44918, a gift from Harry Mobley) for constitutive expression of luciferase and its substrates. EcN transformants were grown on ampicillin and kanamycin supplemented LB agar or in broth as required.

Immunoblotting: To qualitatively confirm plasmid-based expression of FyuA, samples of overnight cultures were grown at 1:100 in fresh antibiotic-supplemented media until the OD600 reached 0.8. Subsequently, the bacteria were centrifuged, the spent media was discarded, and the pellets were washed with PBS (×3). The pellets were lysed with B-PER Bacterial Protein Extraction Reagent (Thermo Fisher Scientific) supplemented with proteases and phosphatases to extract the target proteins for western blot analyses. After quantifying protein concentration via BCA assay, a 1:1 mixture of lysed fraction and Laemmli sample buffer was boiled at 95° C. for 10 min. The proteins were separated by SDS-PAGE and transferred to 0.45-μm Amersham nitrocellulose membranes (GE Healthcare). Membranes were then blocked in 5% milk/TBST for 1 h at room temperature and incubated with Direct-Blot HRP anti-DYKDDDDK (FLAG) tag primary antibody (BioLegend) at 4° C. overnight. Immunoblots were developed using SuperSignal enhanced chemiluminescence (Thermo Fisher Scientific) and imaged via C-DiGit Blot Scanner (LI-COR Biosciences).

Radiolabeling: 10 μL of ⁶⁴Cu or ⁶⁷Cu was mixed with 10 μg (10 μL) of YbT in 0.1 m ammonium acetate (pH 7) to bring the reaction volume to 100 μL. This mixture was vortexed for 10-15 s before incubating in a thermomixer with 800 rpm agitation at 37° C. for 1 h. Radiochemical purity and stability studies were determined using radio-HPLC (Agilent 1260 Infinity II Quaternary System with a Flow-RAM radio-HPLC detector) on a C18 column (Agilent Poroshell 120 EC-C18 column, 3×50 mm, 4 μm) as described before.

Functional Characterization of EcN-fyuA↑: 104-109 cfu of EcNΔfyuA, EcN WT, or EcN-fyuA↑ were incubated with ⁶⁴Cu-YbT (0.04-0.06 Mbq) supplemented LB for 2 h. Subsequently, the samples were centrifuged, and the pellets washed with PBS (×3). Cell-associated ⁶⁴Cu levels were measured from pelleted bacteria using a gamma counter. Experiments were repeated three times from independent bacterial cultures.

Mammalian Cell Culture: Murine breast cancer 4T1 cells (ATCC CRL-2539) and MC38 colon cancer cells (Kerafast #ENH204-FP) were cultured in RPMI 1640 and DMEM respectively containing 10% fetal bovine serum and 5% penicillin-streptomycin solution. All media were purchased from Gibco, Thermo Fisher Scientific. The cells were maintained at 37° C. with 5% CO₂ in air and sub-cultured twice weekly.

Animal Models: 4-8-week-old female BALB/cJ and male C57BL6/J mice (Jackson Laboratory) were used in this study. For tumor induction, 1.5×10⁶ cancer cells (100 μL saline) were injected subcutaneously in the shaved right flank of anesthetized (2% isoflurane) mice. Tumor volume was calculated by measuring the length and width of each tumor every 2-3 days using calipers, where volume=0.5×length×width2. The tumor volume of each mouse was monitored every 2-3 days until the animals reached one of the defined end-points: (i) the longest dimension of tumor>1.2 cm, (ii) tumor became necrotic or ulcerated, (iii) tumor started to hamper movement of the mouse, or (iv) mouse lost>10% of its body weight.

Bacterial strains were grown overnight in LB media, which contained the appropriate antibiotics. A 1:100 dilution into growth media with antibiotics was started the day of injection and grown to an OD600 of ~0.8. Bacteria were centrifuged and washed with PBS (×3) before 5×10⁶ cfu (100 μL saline) were intratumorally injected in mice.

A single dose of 3.7-5.55 Mbq of ⁶⁴Cu-YbT (for imaging) or two fractionated doses of 9.25 Mbq of ⁶⁷Cu-YbT (for therapy) was retro-orbitally administered in each mouse.

For serum analysis, terminal intra-cardiac blood collection was performed. Serum samples were prepared and shipped to IDEXX BioAnalytics for analyses.

All animal experiments were conducted by following a protocol approved by the University of Cincinnati Biosafety, Radiation Safety, and Animal Care and Use Committees (protocol #: 20-05-16-01).

Imaging and Ex Vivo Biodistribution Studies: Small-animal PET scan was performed 24 h post injection of ⁶⁴Cu probes on a μPET scanner (Siemens Inveon). Animals were placed in the supine position on the imaging gantry with continued warming for the duration of the scan. CT scan (80 kVp, 500 μA, at 120 projections) was acquired for anatomical reference overlay with PET image for a 15-min acquisition with real-time reconstruction. PET images were acquired over an additional 15 min and spatial resolution in the entire field of view was determined by ordered subset expectation maximization in 2D. Histogramming and reconstruction were applied using Siemens Inveon software. Post-processing was carried out with Inveon Research Workplace and general analysis was used for contouring volume of interest (VOI). These VOI values were considered active infection volumes and used for further analyses. Bioluminescence images were acquired for 5 min using an IVIS Imaging System for quantification of radiance (total flux, photons per second, p s−1) of the bioluminescent signals from the regions of interest. After the imaging studies, the mice were euthanized via carbon dioxide inhalation and cervical dislocation. Organs and tissues of interest were removed and weighed. Residual radioactivity in the samples was measured with a gamma counter and results expressed as percentage of injected dose per gram of organ (% ID/g). Tumors with bacteria were homogenized and serially diluted in fresh LB media, before plating each dilution on antibiotic supplemented LB agar plates to enumerate the bacteria.

Quantitative PCR with Reverse Transcription: Total DNA extraction from organs and tumors was performed with DNeasy Blood & Tissue Kit (QIAGEN). pGEN-luxCDABE-specific primers (forward: ATGAAATTTGGAAACTTTTTGCTTACATAC and reverse: GGGGTTTACTTTTACCTTATGGAACT) and Luna Universal qPCR Master Mix (NEB) were used to perform qRT-PCR on QuantStudio 3 (Thermo Fisher Scientific) in a 96-well format. Standards were analyzed based on pure bacterial plasmids and used to determine pGEN-luxCDABE concentration from animal tissues.

Flow Cytometry: Harvested tumors were minced into 1 mm pieces with a razor blade and digested in HBSS containing 2 mg/ml Collagenase IV (Gibco) and 20 μg mL-1 DNase I (Sigma-Aldrich) for 45 min at 37° C. under agitation. Tumor suspensions were filtered with 70 μm strainer and debris were removed using the Debris removal solution (Miltenyi) according to the manufacturer's protocol. Five million cells per tumor were used for immunophenotyping by flow cytometry. The single cell suspensions were first labeled with the Fixable Viability Dye eFluor506 (eBioscience) in order to separate live from dead cells during analyses. Fc receptors were blocked by incubation with the mouse FC blocker solution (Miltenyi) for 10 min at 4° C. Samples were incubated 20 min at 4° C. with the following antibodies: anti-CD45-AlexaFluor488 (eBioscience, 30-F11), Anti-CD3-PE-Cy5 (eBioscience, 145-2C11), anti-CD4-APC (eBioscience, RM4-5), anti-CD8a-PerCP-Cy5.5 (eBiosciences, 53-6.7), anti-CD25-PE (eBioscience, PC61.5). Following two washes with PBS the samples were fixed and permeabilized using the Foxp3 staining kit (eBioscience) according to the manufacturer's protocol. Samples were incubated after permeabilization with an anti-Foxp3-PE-Cy7 (eBioscience, FJK-16s) for 30 min at 4° C. Samples were acquired on a BD LSRFortessa 2 in the Cincinnati Children's Hospital Medical Center Flow Cytometry Core. Single stained samples were used to calculate the compensation parameters between the different fluorochrome using Diva software (BD Bioscience). Sample analyses were performed using FlowJo software (BD Bioscience).

Statistical Analyses: Pre-processing of data (e.g., transformation, normalization, and evaluation of outliers) was not performed for any of the experiments unless otherwise stated in the figure legends. All numerical data are presented as mean±s.d. The sample size and statistical methods used for each experiment are stated in detail in the figure legends. All data were analyzed using GraphPad Prism 9.0.0 software, unless noted otherwise.

### Example 1: EcN Internalization of ^{64/67}Cu is Maximized by Overexpression of FyuA

E. coli UTI89 (pathogen) exhibits an approximately threefold higher uptake of ⁶⁴Cu-YbT compared to EcN, a nonpathogen. An FyuA knockout (KO) strain of EcN, EcNΔfyuA, showed minimal uptake of ⁶⁴Cu-YbT in vivo, which was consistent with in vitro analyses from independent studies. Since FyuA facilitates internalization of the metal-siderophore complex, it was postulated that overexpressing FyuA in EcN will maximize uptake of Cu-YbT.

The pSF-OXB20 plasmid was redesigned for the present invention by inserting a FyuA gBlock downstream of the constitutive OXB20 promoter. EcN was thus transformed with the new plasmid of the present invention (pZVS1) to overexpress FyuA (see FIG. 6). Since wild-type (WT) EcN naturally expresses FyuA to some extent, the FLAG tag was relied on to confirm plasmid-based expression of FyuA by the positive transformants (EcN-fyuA↑) (FIG. 1C). Immunoblotting allowed the qualitative confirmation of plasmid-based expression of FyuA. Radioactive Cu-YbT uptake studies were emphasized to prove both expression and functionality of additional FyuA in the engineered probiotic bacterium of the present invention. Variable colony forming units (cfu) of EcNΔfyuA, EcN WT, or EcN-fyuA↑ were incubated in growth media supplemented with the same radioactive amount of ⁶⁴Cu-YbT (0.04-0.06 Mbq) for 2 h (FIG. 1D). Afterward, cell-associated ⁶⁴Cu-YbT was determined for each group of EcN. The underlying assumption was that the total number of FyuA receptors available for ⁶⁴Cu-YbT binding will positively correlate with the bacterial cfu. It was observed that the KO strain accumulated the least amount of ⁶⁴Cu-YbT at all cfu (FIG. 1E). Minimal cell-associated ⁶⁴Cu-YbT in EcNΔfyuA conformed to the results of previous studies and indicated that FyuA is required for bacterial uptake of Cu-YbT. Although EcN WT and EcN-fyuA↑ demonstrated similar uptake at low cfu, the latter had a significantly higher (p<0.05) uptake of the radioactive metal complex when the bacterial population exceeded 107 cfu. Since EcN WT genomically expresses FyuA, it accumulated ⁶⁴Cu-YbT at similar levels to EcN-fyuA↑ when the bacterial population was low, though the latter appeared to outcompete the wild-type strain at cfu that are typically observed in the tumor microenvironment following administration of the engineered probiotic for cancer therapy. Thus, overexpressing FyuA increases cellular uptake of Cu-YbT in EcN. Finally, the dissociation profile of ⁶⁴Cu-YbT was determined from EcN-fyuA↑ over a 24-h period (FIG. 1F). At least 80% of the intact probe was found associated to the cells, which indicates that the genetically encoded E. coli construct bind and retain its cognate Cu-labeled probe, with high specificity and affinity. Data in FIG. 1E and FIG. 1F presented as mean±s.d. (n=3); data in FIG. 1E (109 cfu) analyzed by one-way ANOVA and Dunnett's T3 multiple comparison test with Brown-Forsythe and Welch's correction, alpha=0.05. * p<0.05, ** p<0.01.

### Example 2: Engineered EcN Colonize and Persist in the Tumor Microenvironment

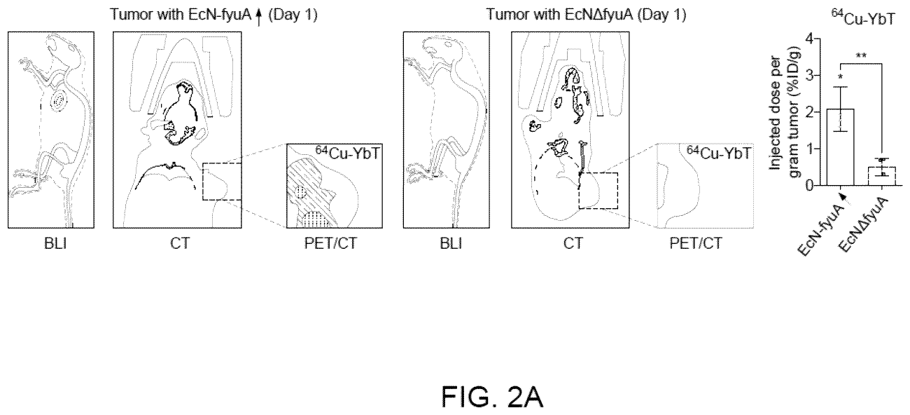

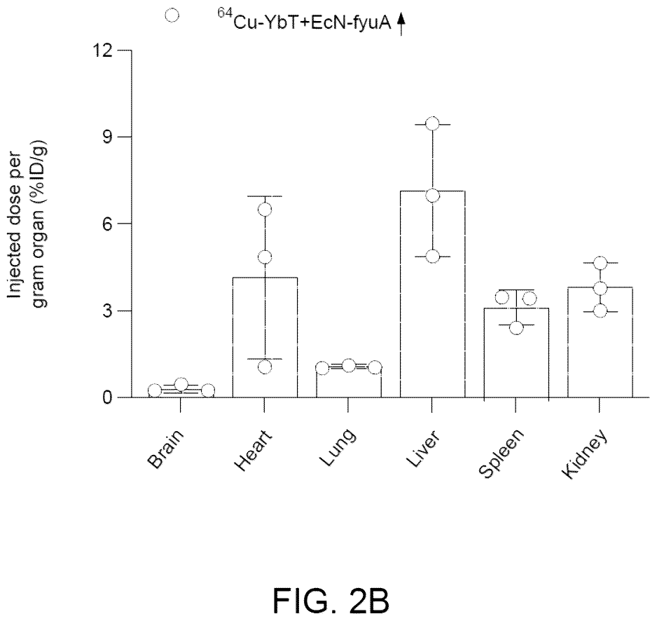

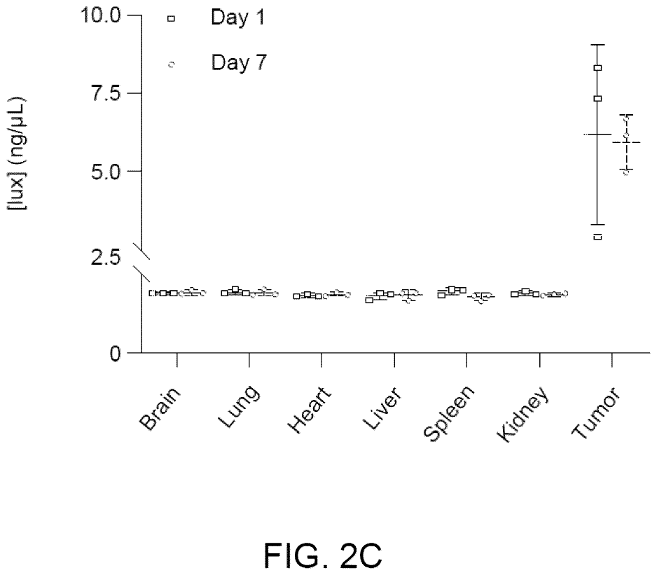

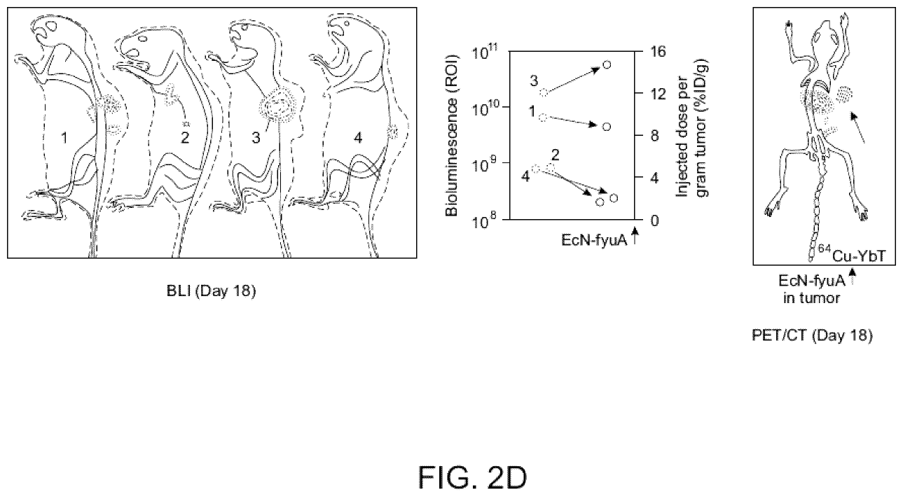

Imaging studies were performed to recapitulate the in vitro observations in vivo. EcN-fyuA↑ and EcNΔfyuA were administered intratumorally in a syngeneic MC38/C57BL6 colon cancer model. Then, ⁶⁴Cu-YbT was administered retro-orbitally for PET/CT imaging. Transformation of EcN-fyuA↑ with pGEN-luxCDABE (luciferase) allowed for confirmation of bacterial localization in the tumor via bioluminescence imaging (BLI) (FIG. 2A). The data of the bar-chart in FIG. 2A is presented as mean±s.d. (n=3-4) analyzed by Welch's t-test. ** p<0.01.

Following PET/CT imaging, significantly higher signals were noticed in tumors with EcN-fyuA↑ compared to those with EcNΔfyuA. Ex vivo biodistribution (BioD) analyses also revealed statistically pronounced (p<0.01) accumulations of ⁶⁴Cu-YbT in tumors hosting FyuA-overexpressing EcN. In accordance with the previous observations, the probe was cleared primarily by the liver and kidneys with minimal accumulation in rest of the major organs (FIG. 2B).

To determine the extent of colonization, persistence, and growth of intratumorally administered bacteria, longitudinal genomic, cell, and imaging analysis was performed. EcN-fyuA↑-lux was administered in MC38 tumors before the mice were euthanized and the major organs harvested to check for bacterial presence 1- and 7-days post-administration via quantitative real-time polymerase chain reaction (qRT-PCR). Using lux-specific primers, significant bacterial presence was confirmed exclusively in the tumors at both time-points (FIG. 2C). All readings for the major organs were below the limit of detection for the analysis, which was 2.5 ng μL-1. Moreover, the qRT-PCR analyses indicated negligible bacterial extravasation out of tumors, thus preventing localization in off-target tissues, which is consistent with previous reports of engineered EcN constructs. A separate cohort of mice was used to determine EcN-fyuA↑ population in the tumors 2-days after bacterial administration. After euthanizing the mice, the tumors were disaggregated and serial dilutions of the homogenates were plated on antibiotic supplemented LB plates. It was observed that there were approximately ten times more bacteria (5.9±2.8×107 cfu) in the tumors than the amount injected (5×106 cfu) 48 h before. Then, imaging was performed to check for microbial presence on day 18, when the tumor volume was fairly large. BLI revealed varying levels of EcN-fyuA↑, which correlated positively with the amount of ⁶⁴Cu-YbT accumulation in the tumors (FIG. 2D). PET/CT imaging corroborated the fact that the engineered bacteria are not only able to maintain their population inside solid tumors, but also achieve a sustained growth pattern, consistent with tumor growth.

### Example 3: EcN-fyuA↑ Concentrates ⁶⁷Cu-YbT in Solid Tumors to Elicit Anti-Tumor Effects

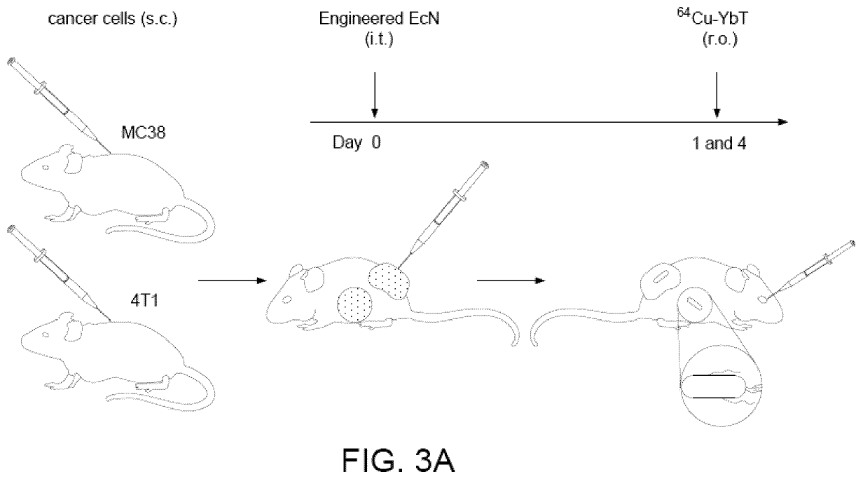

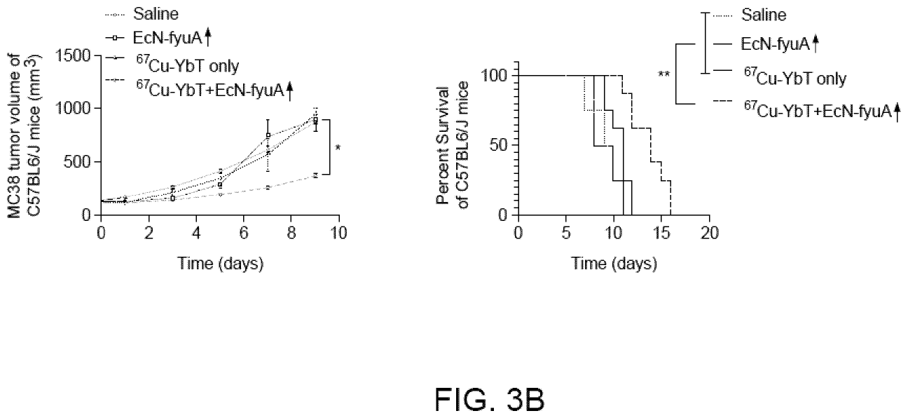

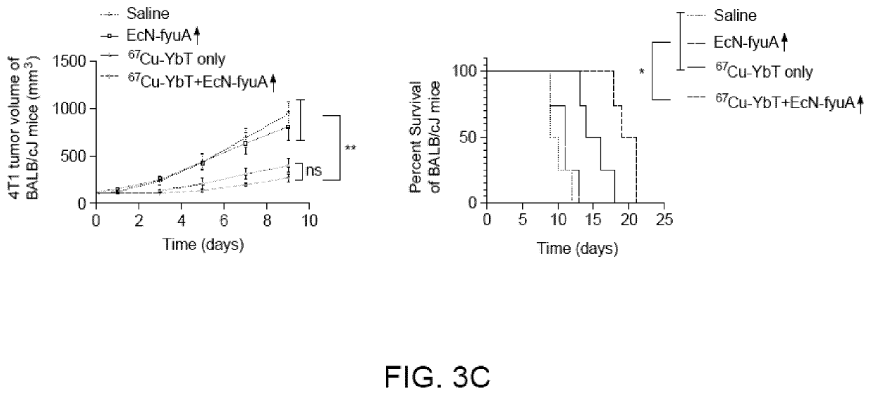

After validating the specific interactions of EcN-fyuA↑ with ⁶⁴Cu-YbT, the high energy β-emitting radiotherapeutic isotope, ⁶⁷Cu, was used instead. The ligand as radiolabeled using the same technique used for ⁶⁴Cu. The yield and purity of ⁶⁷Cu-YbT was >95%. The stability of the probe was >80% over a 24-h period in mouse serum. For therapy studies, two different murine subcutaneous tumor models were used—colon cancer MC38 and breast cancer 4T1 (FIG. 3A). Saline was injected, or EcN-fyuA↑ intratumorally on day 0, followed by saline or two fractionated doses of ⁶⁷Cu-YbT on day 1 and 4 retro-orbitally. For both tumor models, the groups of mice that received a combination of EcN-fyuA↑ with ⁶⁷Cu-YbT survived significantly longer than those that received saline, bacteria only, or ⁶⁷Cu-YbT only (FIGS. 3B and 3C). Regarding FIGS. 3B and 3C, data in tumor growth curves is presented as mean±s.d.; the final tumor volumes were analyzed by one-way ANOVA and Dunnett's T3 multiple comparison test with Brown-Forsythe and Welch's correction, alpha=0.05. Survival curves were analyzed by Kaplan-Meier with log-rank (Mantel-Cox) test by comparing two groups at a time and presenting the p-value at which ⁶⁷Cu-YbT+EcN-fyuA↑ is significantly different from the other three treatment strategies. ns=not significant, *p<0.05, ** p<0.01.

In C57BL6/J model, the combination treatment extended the median survival of mice with highly aggressive MC38 tumors from 8 days in the control groups to 13 days, after initiation of treatment. On the other hand, the median survival in 4T1 tumor-bearing mice, also a syngeneic highly aggressive model, improved from 11 days in the control groups to 18 days in the combination group (though not statistically significantly when compared to all control groups together). Importantly, EcN-fyuA↑+⁶⁷Cu-YbT halted tumor progression significantly more than ⁶⁷Cu-YbT alone (MC38: p<0.05). This conforms to the PET/CT imaging data and indicates that the presence of the engineered microbe in the tumor microenvironment allowed higher retention of therapeutic ⁶⁷Cu to attenuate tumor growth, in the colon cancer model.

### Example 4: EcN-fyuA↑ and ⁶⁷Cu-YbT Remodels Immune Landscape of Tumors without Systemic Toxicity

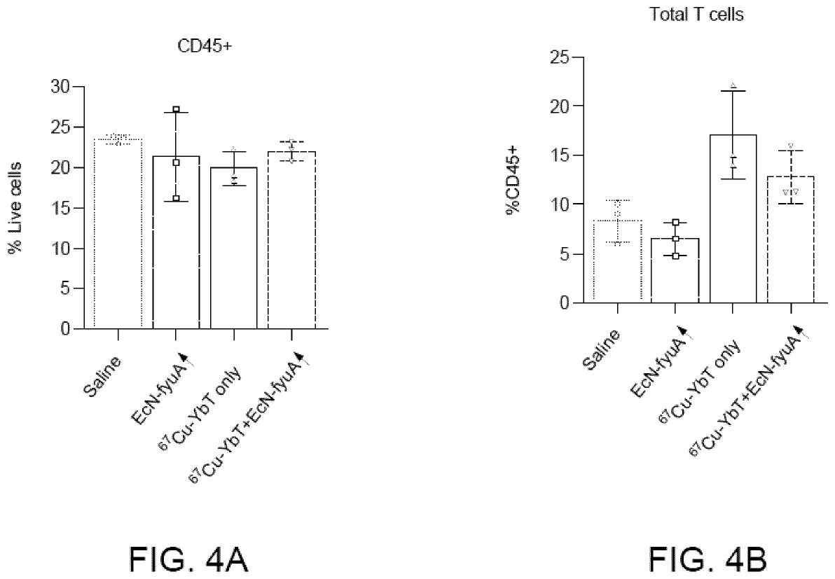

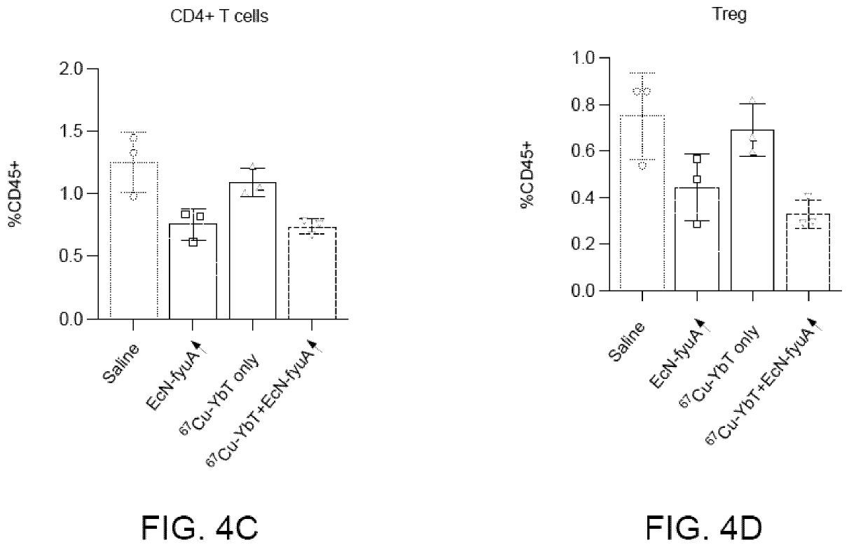

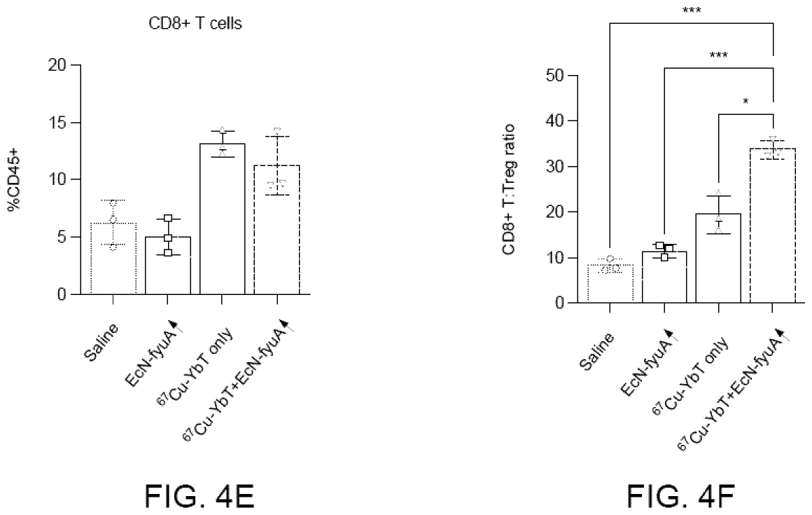

Next, the immune cell profile of the MC38 tumor microenvironment was evaluated 7 days post-treatment. Based on preliminary screening there were no significant differences in total immune cell infiltrates (CD45+) (FIG. 4A), though the global T cell (FIG. 4B) tumor infiltration appeared to increase following ⁶⁷Cu-YbT administration. Further analyses revealed that CD4+ T cells (FIG. 4C), which typically activate macrophages and B cells to clear extracellular pathogens, decreased in tumors that contained EcN-fyuA↑. This likely indicates that the immune cells do not recognize EcN-fyuA↑ as a nonself-antigen and organism. This might also explain the sustained presence of the bacteria in the tumor microenvironment (FIGS. 2C and 2D). On the other hand, the drop in CD4+ T cell population might be attributed to the concordant reduction in its subset, the regulatory T cell (Treg) population (FIG. 4D). Cytotoxic CD8+ T cell (FIG. 4E) infiltration increased following systemic administration of ⁶⁷Cu-YbT regardless of the presence of bacteria. This indicates that ⁶⁷Cu-YbT, during its transit in the tumor microenvironment, elicited sufficient mutations in cancer cells for them to present neoantigens to activate CD8+ T cells. This resulted in a surge in cytotoxic T cell population to clear the malignant cells. Importantly, the simultaneous egress of immunosuppressive Treg cells meant that the CD8+T:Treg cell ratio was significantly higher in tumors of mice that received a combination of EcN-fyuA↑ and ⁶⁷Cu-YbT compared to all other three groups combined (FIG. 4F). This phenomenon has been observed in a recent TRT investigation as well, thus affirming EcN-guided ⁶⁷Cu-YbT delivery to solid tumors as a potential microbe-based pretargeted immunomodulatory TRT platform. Regarding FIGS. 4A-4F, all data is presented as mean±s.d. (n=3). The data in FIG. 4F was analyzed by one-way ANOVA and Dunnett's T3 multiple comparison test with Brown-Forsythe and Welch's correction, alpha=0.05. * p<0.05, ** p<0.01, *** p<0.001.

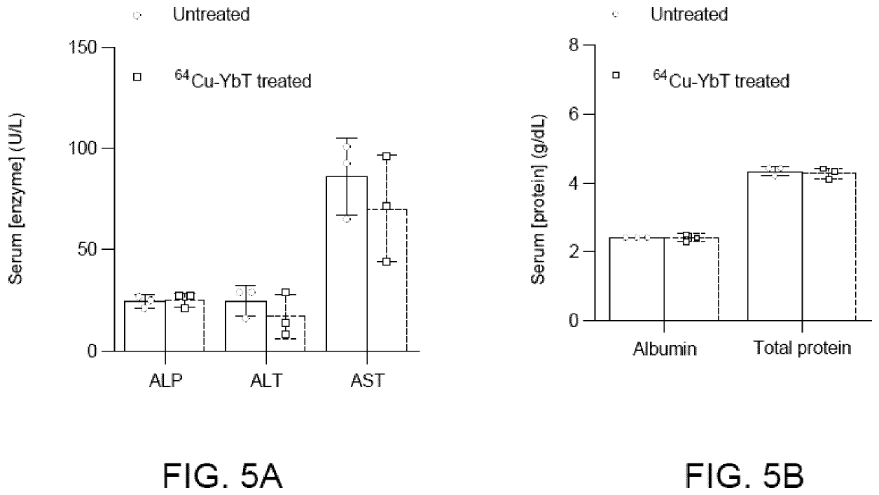

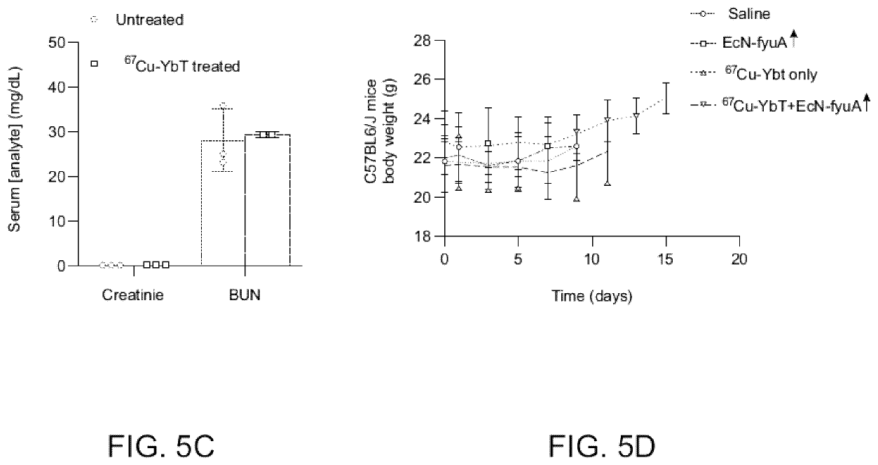

There are several reports that show that EcN has good preclinical biocompatibility. Additionally, the present data shows (FIG. 2D) that the engineered bacteria of the present invention, which is administered intratumorally, remain primarily confined in the solid tumors with no evidence of localizing in metabolic organs like the liver or kidneys. However, ⁶⁷Cu-YbT, which is administered systemically is likely to be cleared by liver and kidneys to some extent as revealed by the PET imaging. Therefore, it was pertinent to evaluate the safety of the radiopharmaceutical. In order to determine whether ⁶⁷Cu-YbT negatively alters hepatic and renal function in mice, serum samples were analyzed from mice treated with and without ⁶⁴Cu-YbT, using standard toxicity screening. Hepatic enzyme analyses did not reveal any significant differences in alanine transaminase, alkaline phosphatase, or aspartate transaminase between the two groups of mice (FIGS. 5A and 5B). Bilirubin and total protein concentrations did not vary between the two treatment groups either. Since liver damage is typically associated with elevated levels of metabolic enzymes and total protein concentrations in the serum, it can be inferred that ⁶⁷Cu-YbT did not result in off-target liver toxicity in the mice. Likewise, with renal function no changes were observed in creatinine concentrations and blood urea nitrogen levels from normal values (FIG. 5C). Finally, to assess the overall wellbeing of the mice, their weights were monitored every 2 days. While the general trend was a slight increase in body weight, which was attributed to progressive tumor growth, the differences were not statistically significant (FIG. 5D). Regarding FIGS. 5A-5D, the data is presented as mean±s.d. (n=3).

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

SEQUENCES
SEQ ID NO: 1
GCGATCGCGGCTCCCGACATCTTGGACCATTAGCTCCACAGGTATC
TTCTTCCCTCTAGTGGTCATAACAGCAGCTTCAGCTACCTCTCAA
TTCAAAAAACCCCTCAAGACCCGTTTAGAGGCCCCAAGGGGTTAT
GCTATCAATCGTTGCGTTACACACACAAAAAACCAACACACATCC
ATCTTCGATGGATAGCGATTTTATTATCTAACTGCTGATCGAGTG
TAGCCAGATCTAAGCTGTTGTGACCGCTTGCTCTAGCCAGCTATC
GAGTTGTGAACCGATCCATCTAGCAATTGGTCTCGATCTAGCGAT
AGGCTTCGATCTAGCTATGTAGAAACGCCGTGTGCTCGATCGCTT
GATAAGGTCCACGTAGCTGCTATAATTGCTTCAACAGAACATATT
GACTATCCGGTATTACCCGGCAGATCTTTGTCGATCCTACCATCC
ACTCGACACACCCGCCAGCGGCCGAGGAGGTAATATGAAAATGAC
ACGGCTTTATCCTCTGGCCTTGGGGGGATTATTGCTCCCCGCCAT
TGCTAATGCCCAGACTTCACAGCAAGACGAAAGCACGCTGGTGGT
TACCGCCAGTAAACAATCTTCCCGCTCGGCATCAGCCAACAACGT
CTCGTCTACTGTTGTCAGCGCGCCGGAATTAAGCGACGCCGGCGT
CACCGCCAGCGACAAACTCCCCAGAGTCTTGCCCGGGCTCAATAT
TGAAAATAGCGGCAACATGCTTTTTTCGACGATCTCGCTACGCGG
CGTCTCTTCAGCGCAGGACTTCTATAACCCCGCCGTCACCCTGTA
TGTCGATGGCGTCCCTCAGCTTTCCACCAACACCATCCAGGCGCT
TACCGATGTGCAAAGCGTGGAGTTGCTGCGAGGCCCACAGGGAAC
GTTATATGGCAAAAGCGCTCAGGGCGGGATCATCAACATCGTCAC
CCAGCAGCCGGACAGCACGCCGCGCGGCTATATTGAAGGCGGCGT
CAGTAGCCGCGACAGTTATCGAAGTAAGTTCAACCTGAGCGGCCC
CATTCAGGATGGCCTGCTGTACGGCAGCGTCACCCTGTTACGCCA
GGTTGATGACGGCGACATGATTAACCCCGCGACGGGAAGCGATGA
CTTAGGCGGCACCCGCGCCAGCATAGGGAATGTGAAACTGCGTCT
GGCGCCGGACGATCAGCCCTGGGAAATGGGCTTTGCCGCCTCACG
CGAATGTACCCGCGCCACCCAGGACGCCTATGTGGGATGGAATGA
TATTAAGGGCCGTAAGCTGTCGATCAGCGATGGTTCACCAGACCC
GTACATGCGGCGCTGCACTGACAGCCAGACCCTGAGTGGGAAATA
CACCACCGATGACTGGGTTTTCAACCTGATCAGCGCCTGGCAGCA
GCAGCATTATTCGCGCACCTTCCCTTCCGGTTCGTTAATCGTCAA
TATGCCTCAGCGCTGGAATCAGGATGTGCAGGAGCTGCGCGCCGC
AACCCTGGGCGATGCGCGTACCGTTGATATGGTGTTTGGGCTGTA
CCGGCAGAACACCCGCGAGAAGTTAAATTCAGCCTACGACATGCC
GACAATGCCTTATTTAAGCAGTACCGGCTATACCACCGCTGAAAC
GCTGGCCGCATACAGTGACCTGACCTGGCATTTAACCGATCGTTT
TGATATCGGCGGCGGCGTGCGCTTCTCGCATGATAAATCCAGTAC
ACAATATCACGGCAGCATGCTCGGCAACCCGTTTGGCGACCAGGG
TAAGAGCAATGACGATCAGGTGCTCGGGCAGCTATCCGCAGGCTA
TATGCTGACCGATGACTGGAGAGTGTATACCCGTGTAGCCCAGGG
ATATAAACCTTCCGGGTACAACATCGTGCCTACTGCGGGTCTTGA
TGCCAAACCGTTCGTCGCCGAGAAATCCATCAACTATGAACTTGG
CACCCGCTACGAAACCGCTGACGTCACGCTGCAAGCCGCGACGTT
TTATACCCACACCAAAGACATGCAGCTTTACTCTGGCCCGGTCGG
GATGCAGACATTAAGCAATGCGGGTAAAGCCGACGCCACCGGCGT
TGAGCTTGAAGCGAAGTGGCGGTTTGCGCCAGGCTGGTCATGGGA
TATCAATGGCAACGTGATCCGTTCCGAATTCACCAATGACAGTGA
GTTGTATCACGGTAACCGGGTGCCGTTCGTACCACGTTATGGCGC
GGGAAGCAGCGTGAACGGCGTGATTGATACGCGCTATGGCGCACT
GATGCCCCGACTGGCGGTTAATCTGGTCGGGCCGCATTATTTCGA
TGGCGACAACCAGTTGCGGCAAGGCACCTATGCCACCCTGGACAG
CAGCCTGGGCTGGCAGGCGACTGAACGGATGAACATTTCCGTCTA
TGTCGATAACCTGTTCGACCGTCGTTACCGTACCTATGGCTACAT
GAACGGCAGCAGCGCCGTCGCGCAGGTCAATATGGGTCGCACCGT
CGGTATCAATACGCGAATTGATTTCTTCGTCGACGAAAACCTGTA
CTTCCAAGGTGACTACAAGGACGATGACGATAAGTGGAGCCATCC
GCAGTTTGAGAAATGAAAGCTTCCGAGCTCTCGAATTCAAAGGAG
GTACCCACCATGGGGTACCGCGATATCTACCTCGAGGTTTCTAGA
AGTTGTCTCCTCCTGCACTGACTGACTGATACAATCGATTTCTGG
ATCCGCAGGCCTCTGCTAGCTTGACTGACTGAGATACAGCGTACC
TTCAGCTCACAGACATGATAAGATACATTGATGAGTTTGGACAAA
CCACAACTAGAATGCAGTGAAAAAAATGCTTTATTTGTGAAATTT
GTGATGCTATTGCTTTATTTGTAACCATTATAAGCTGCAATAAAC
AAGTTAACAACAACAATTGCATTCATTTTATGTTTCAGGTTCAGG
GGGAGGTGTGGGAGGTTTTTTAAAGCAAGTAAAACCTCTACAAAT
GTGGTATTGGCCCATCTCTATCGGTATCGTAGCATAACCCCTTGG
GGCCTCTAAACGGGTCTTGAGGGGTTTTTTGTGCCCCTCGGGCCG
GATTGCTATCTACCGGCATTGGCGCAGAAAAAAATGCCTGATGCG
ACGCTGCGCGTCTTATACTCCCACATATGCCAGATTCAGCAACGG
ATACGGCTTCCCCAACTTGCCCACTTCCATACGTGTCCTCCTTAC
CAGAAATTTATCCTTAAGGTCGTCAGCTATCCTGCAGGCGATCTC
TCGATTTCGATCAAGACATTCCTTTAATGGTCTTTTCTGGACACC
ACTAGGGGTCAGAAGTAGTTCATCAAACTTTCTTCCCTCCCTAAT
CTCATTGGTTACCTTGGGCTATCGAAACTTAATTAACCAGTCAAG
TCAGCTACTTGGCGAGATCGACTTGTCTGGGTTTCGACTACGCTC
AGAATTGCGTCAGTCAAGTTCGATCTGGTCCTTGCTATTGCACCC
GTTCTCCGATTACGAGTTTCATTTAAATCATGTGAGCAAAAGGCC
AGCAAAAGGCCAGGAACCGTAAAAAGGCCGCGTTGCTGGCGTTTT
TCCATAGGCTCCGCCCCCCTGACGAGCATCACAAAAATCGACGCT
CAAGTCAGAGGTGGCGAAACCCGACAGGACTATAAAGATACCAGG
CGTTTCCCCCTGGAAGCTCCCTCGTGCGCTCTCCTGTTCCGACCC
TGCCGCTTACCGGATACCTGTCCGCCTTTCTCCCTTCGGGAAGCG
TGGCGCTTTCTCATAGCTCACGCTGTAGGTATCTCAGTTCGGTGT
AGGTCGTTCGCTCCAAGCTGGGCTGTGTGCACGAACCCCCCGTTC
AGCCCGACCGCTGCGCCTTATCCGGTAACTATCGTCTTGAGTCCA
ACCCGGTAAGACACGACTTATCGCCACTGGCAGCAGCCACTGGTA
ACAGGATTAGCAGAGCGAGGTATGTAGGCGGTGCTACAGAGTTCT
TGAAGTGGTGGCCTAACTACGGCTACACTAGAAGAACAGTATTTG
GTATCTGCGCTCTGCTGAAGCCAGTTACCTTCGGAAAAAGAGTTG
GTAGCTCTTGATCCGGCAAACAAACCACCGCTGGTAGCGGTGGTT
TTTTTGTTTGCAAGCAGCAGATTACGCGCAGAAAAAAAGGATCTC
AAGAAGATCCTTTGATCTTTTCTACGGGGTCTGACGCTCAGTGGA
ACGAAAACTCACGTTAAGGGATTTTGGTCATGAGATTATCAAAAA
GGATCTTCACCTAGATCCTTTTAAATTAAAAATGAAGTTTTAAAT
CAATCTAAAGTATATATGAGTAAACTTGGTCTGACAGTTACCAAT
GCTTAATCAGTGAGGCACCTATCTCAGCGATCTGTCTATTTCGTT
CATCCATAGTTGCATTTAAATTTCCGAACTCTCCAAGGCCCTCGT
CGGAAAATCTTCAAACCTTTCGTCCGATCCATCTTGCAGGCTACC
TCTCGAACGAACTATCGCAAGTCTCTTGGCCGGCCTTGCGCCTTG
GCTATTGCTTGGCAGCGCCTATCGCCAGGTATTACTCCAATCCCG
AATATCCGAGATCGGGATCACCCGAGAGAAGTTCAACCTACATCC
TCAATCCCGATCTATCCGAGATCCGAGGAATATCGAAATCGGGGC
GCGCCTGGTGTACCGAGAACGATCCTCTCAGTGCGAGTCTCGACG
ATCCATATCGTTGCTTGGCAGTCAGCCAGTCGGAATCCAGCTTGG
GACCCAGGAAGTCCAATCGTCAGATATTGTACTCAAGCCTGGTCA
CGGCAGCGTACCGATCTGTTTAAACCTAGATATTGATAGTCTGAT
CGGTCAACGTATAATCGAGTCCTAGCTTTTGCAAACATCTATCAA
GAGACAGGATCAGCAGGAGGCTTTCGCATGATTGAACAAGATGGA
TTGCACGCAGGTTCTCCGGCGGCTTGGGTGGAGAGGCTATTCGGC
TATGACTGGGCACAACAGACAATCGGCTGCTCTGATGCCGCCGTG
TTCCGGCTGTCAGCGCAGGGGCGTCCGGTTCTTTTTGTCAAGACC
GACCTGTCCGGTGCCCTGAATGAACTGCAAGACGAGGCAGCGCGG
CTATCGTGGCTGGCGACGACGGGCGTTCCTTGCGCGGCTGTGCTC
GACGTTGTCACTGAAGCGGGAAGGGACTGGCTGCTATTGGGCGAA
GTGCCGGGGCAGGATCTCCTGTCATCTCACCTTGCTCCTGCCGAG
AAAGTATCCATCATGGCTGATGCAATGCGGCGGCTGCATACGCTT
GATCCGGCTACCTGCCCATTCGACCACCAAGCGAAACATCGCATC
GAGCGAGCACGTACTCGGATGGAAGCCGGTCTTGTCGATCAGGAT
GATCTGGACGAAGAGCATCAGGGGCTCGCGCCAGCCGAACTGTTC
GCCAGGCTCAAGGCGTCTATGCCCGACGGCGAGGATCTCGTCGTG
ACCCACGGCGATGCCTGCTTGCCGAATATCATGGTGGAAAATGGC
CGCTTTTCTGGATTCATCGACTGTGGCCGTCTGGGTGTGGCGGAC
CGCTATCAGGACATAGCGTTGGCTACCCGTGATATTGCTGAAGAG
CTTGGCGGCGAATGGGCTGACCGCTTCCTTGTGCTTTACGGTATC
GCCGCGCCCGATTCGCAGCGCATCGCCTTCTATCGCCTTCTTGAC
GAGTTCTTCTGACCGATTCTAGGTGCATTGGCGCAGAAAAAAATG
CCTGATGCGACGCTGCGCGTCTTATACTCCCACATATGCCAGATT
CAGCAACGGATACGGCTTCCCCAACTTGCCCACTTCCATACGTGT
CCTCCTTACCAGAAATTTATCCTTAAGGTCGTTTAAACTCGACTC
TGGCTCTATCGAATCTCCGTCGTTTCGAGCTTACGCGAACAGCCG
TGGCGCTCATTTGCTCGTCGGGCATCGAATCTCGTCAGCTATCGT
CAGCTTACCTTTTTGGCA

What is claimed is:

1. A genetically engineered Escherichia coli Nissle 1917 bacterium comprising an overexpressed number of FyuA receptors.

2. The genetically engineered bacterium of claim 1 comprising the nucleotide sequence shown in SEQ ID NO:1.

3. The genetically engineered bacterium of claim 1 further comprising cell-associated ⁶⁷Cu complexed to yersiniabactin (⁶⁷Cu-YbT).

4. The genetically engineered bacterium of claim 1 further comprising cell-associated ⁶⁴Cu complexed to yersiniabactin (⁶⁴Cu-YbT).

5. A pharmaceutical composition comprising the genetically engineered bacterium of claim 1 and a pharmaceutically acceptable excipient.

6. A method of providing targeted radionuclide therapy to subject with a tumor having cancer cells comprising administering to the subject a bacterium that has been genetically engineered to have an overexpressed number of FyuA receptors; wherein the bacterium further comprises at least one cell-associated copper radioisotope; and further, wherein the bacterium populates the tumor and delivers a cytotoxic dose of the radioisotope to the cancer cells.

7. The method of claim 6 wherein the copper radioisotope is ⁶⁷Cu.

8. The method of claim 7 wherein the copper radioisotope is complexed to yersiniabactin (YbT).

9. The method of claim 6 wherein the bacterium is genetically engineered Escherichia coli Nissle 1917.

10. The method of claim 6 wherein the bacterium comprises the nucleotide sequence shown in SEQ ID NO:1.

11. A method of imaging a tumor in subject with a tumor comprising administering to the subject a bacterium that has been genetically engineered to have an overexpressed number of FyuA receptors; wherein the bacterium further comprises at least one cell-associated copper radioisotope; wherein the bacterium populates the tumor, and further, wherein positron emission tomography (PET) imaging is conducted on the subject after the bacterium has populated the tumor.

12. The method of claim 11 wherein the copper radioisotope is ⁶⁴Cu.

13. The method of claim 12 wherein the copper radioisotope is complexed to yersiniabactin (YbT).

14. The method of claim 11 wherein the bacterium is genetically engineered Escherichia coli Nissle 1917.

15. The method of claim 11 wherein the bacterium comprises the nucleotide sequence shown in SEQ ID NO:1.
